(19) United States
(12) Patent Application Publication
(10) Pub. No.: US 2017/0312313 A1
(43) Pub. Date: Nov. 2, 2017

(54) SYNTHESIS OF ULTRA-SMALL CERIA-ZIRCONIA NANOPARTICLES AND CERIA-ZIRCONIA NANO COMPLEX AND ITS APPLICATION AS A THERAPEUTIC AGENT FOR SEPSIS

(71) Applicants: Seoul National University R&DB Foundation, Seoul (KR); INSTITUTE FOR BASIC SCIENCE, Daejeon (KR)

(72) Inventors: Taeg Hwan HYEON, Seoul (KR); Seung Hoon LEE, Seoul (KR); Min SOH, Seoul (KR); Chi Kyung KIM, Seoul (KR)

(73) Assignees: Seoul National University R&DB Foundation, Seoul (KR); INSTITUTE FOR BASIC SCIENCE, Daejeon (KR)

(21) Appl. No.: 15/145,823

(22) Filed: May 4, 2016

(30) Foreign Application Priority Data
Apr. 28, 2016 (KR) 10-2016-0052102

(51) Int. Cl. A61K 33/24; A61K 9/51

(52) U.S. Cl. CPC A61K 33/24; A61K 9/5146

(57) ABSTRACT

Disclosed is a ceria-zirconia nanoparticles comprising a core layer consisting of particles made of ceria-zirconia; and a surfactant layer formed by binding a surfactant on the surface of the core layer so as to easily react in vivo, and more particularly, to applying a ceria-zirconia nano complex to an application field as an activator and a sepsis treating agent.

### CROSS-REFERENCE TO RELATED APPLICATION

This application claims priority to and the benefit of Korean Patent Application No. 10-2016-0052102, filed on Apr. 28, 2016, the disclosure of which is incorporated herein by reference in its entirety.

### BACKGROUND OF THE INVENTION

### Field of the Invention

The present invention relates to a synthesis of ceria-zirconia nanoparticles and a ceria-zirconia nano complex having a phospholipid-polyethylene glycol (PEG) layer so as to easily react in vivo, and more particularly, to applying a ceria-zirconia nano complex to an application field as an activator and a sepsis treating agent.

### Description of the Related Art

Ceria (CeO₂) nanoparticles have been mainly used as catalysts of chemical reaction. The ceria nanoparticles have a self-catalyst function and may maintain anti-oxidation for a long time like enzymes. However, the efficiency is high and toxicity is strong, and thus, concerns about side effects are large and it is difficult to graft the ceria nanoparticles onto biomedicine.

Peritonitis is one of diseases in which surgical treatment needs to be urgently performed, and may proceed to sepsis when microbes are invaded into the blood and appropriate treatment or surgery is not performed for a short time. The sepsis is one of systemic inflammatory response syndromes (SIRS) while the body is infected with the microorganisms and a serious systemic inflammatory response appears. It is important to treat organ infection that causes sepsis and an infected site of the body causing the sepsis is found through physical examination, blood inspection, and imaging inspection and then treated with an appropriate antibiotic. When the appropriate treatment is not performed, it can lead to death, and when malfunction, shock, or the like of the human organ function is associated, the death rate is very high. Accordingly, in the actual treatment process, inflammation and reactive oxygen species (ROS) need to be prevented from being generated before surgery according to peritonitis symptoms. However, it is difficult to perform detailed initial response treatment to a peritonitis patient.

In the Journal of the American Medical Association (JAMA) in 2016, the sepsis is newly defined as “an organ malfunction that threatens life caused by dysregulation of the body to the infection’. The infection by the microorganisms occurs and as a result, the organ dysfunction is generated due to excessive inflammation-increased release of cytokines, increased vascular permeability, sympathetic nervous reactivity, and the like. In the sepsis, adverse reaction of the body becomes a larger problem than the infection itself, and in a serious case, the septic shock state is in progress to lead to the death due to multiple organ dysfunction. The death rate of the sepsis caused in the hospital is less than 10% and the death rate due to the septic shock reaches 40%. When the adverse reaction of the body occurs once to the sepsis patient, due to increased vascular permeability, metabolic acidosis (in a state where the blood is in an acidic state by increasing an acidic material other than carbon dioxide beyond buffer capacity), a reduction of tissue perfusion, and the like, the organ dysfunction is continuous in progress even though adjusted with antibiotics. In order to correct the organ dysfunction, various medical efforts such as appropriate fluids supply, acidosis correction, administration of booster, hemodialysis if necessary, and the like are required. That is, the most important factor of prognosis among the treatment methods for the sepsis is how rapidly the antibiotic starts and a method of blocking the infection before the body averse reaction is largely spread is most important. Then, in spite of medical development, the sepsis has a golden time which should not miss the clinical (an initial important time for rescuing a patient from the disease) and there is no treatment method capable of reducing the excessive body adverse reaction itself.

A conversion reaction between Ce³⁺ and Ce⁴⁺ continuously occurs on the surface of the ceria nanoparticles and serves to reinforce a catalyst action of the ceria nanoparticles. In this case, when other metal ions are added to the ceria nanoparticles, the reaction occurring on the surface of the ceria nanoparticles may be controlled and the antioxidation of the ceria nanoparticles may be improved. The zirconium ions are one of metal ions capable of controlling the reaction on the surface of the ceria nanoparticles and improving antioxidation.

In the related art, it was known that when introducing zirconium ions to the ceria nanoparticles, the ceria nanoparticles increases stability at a high temperature and improves reactivity as the catalyst. However, generally, the applied field is limited only to manufacturing and industrial fields and the like.

In International Publication No. PCT/EP2012/063756, it is mentioned that a mixed oxide including cerium and zirconium oxides and a preparing method thereof are published and the composition may be used as a catalyst in vehicle engine fuel. However, in PCT/EP2012/063756, a use for antioxidant or treating sepsis treating and the like are not described at all by using the ceria-zirconia nanoparticles and the like.

Accordingly, in the present invention, ceria-zirconia nanoparticles introducing zirconium ions to the ceria nanoparticles have more excellent effects on antioxidation and anti-inflammatory than the existing ceria nanoparticles and side effects on toxicity may be minimized by adjusting the reactivity on the surface of the ceria nanoparticles. Further, the effect on the antioxidation and the anti-inflammatory is increased due to an increase in a surface area to volume as the size of the nanoparticles is smaller and uniform.

Furthermore, as an in vivo experiment for treating and preventing sepsis, the antioxidation effect of the ceria-zirconia nanoparticles is applied to the actual sepsis disease model to have an effect for treating and preventing, and thus, the present invention is completed.

### CITATION LIST

### Patent Literature

Patent Literature 1: International Patent Publication No. PCT/EP2012/063756, ‘Ceria zirconia alumina composition with enhanced thermal stability’

### SUMMARY OF THE INVENTION

Sepsis is one of diseases having high death rate when acutely progressing, and in order to suppress the acutely progressing process, a material having antioxidant effect is required. Accordingly, synthesized uniform nanoparticles having very small sizes may be used as an activator by introducing zirconium ions to existing ceria nanoparticles due to an excellent effect of removing reactive oxygen species (ROS). Further, as the size of the particle is decreased, the surface area to volume is increased and reactivity may be increased. Accordingly, death rate due to acute sepsis may be further lowered as compared with an existing treating agent by using the ceria-zirconia nanoparticles having small sizes.

A technical object to be achieved in the present invention is not limited to the aforementioned technical objects, and another not-mentioned technical object will be obviously understood by those skilled in the art from the description below.

In one aspect, the present invention provides ceria-zirconia nanoparticles comprising a core layer consisting of particles made of ceria-zirconia and a surfactant layer formed by binding a surfactant on the surface of the core layer.

Cerium is a metal element having large chemical reactivity and mainly has an oxidation state of Ce³⁺ or Ce⁴⁺ in a compound, and generally, the Ce³⁺ state is more stable, but in the case of oxide, the Ce⁴⁺ state is more stable. Cerium in the oxidation state of Ce³⁺ is called cerium (III) and cerium of Ce⁴⁺ is called cerium (IV). Cerium oxide is two of cerium (III) oxide (Ce₂O₃) and cerium (IV) oxide (CeO₂) and at room temperature under atmosphere pressure, CeO₂ called ceria is more stable.

Zirconium is silver-gray transition metal belonging to group IV (group 4B) called a titanium group. The metal itself has large reactivity and zirconium dioxide (ZrO₂) called zirconia is a stable material having low reactivity.

The particle means an object having a minute size configuring the material, and particularly, the ceria-zirconia nanoparticles mean a nano-sized material of a single crystal constituted by a solid mixture in which ceria and zirconia form a completely uniform phase.

In a preferred embodiment, the core layer may have a diameter of 1 to 5 nm.

In another preferred embodiment, the ceria-zirconia may be constituted by CeₓZr₁-xO₂ and x may be 0.1 to 1.

In still another preferred embodiment, x may be 0.7.

In yet another preferred embodiment, the surfactant may be at least one selected from a group consisting of C₆-C₂₀ alkyl amine, alkyl trimethyl ammonium salt ((CH₃)₃RNX, wherein R is C₈-C₂₅, X is Br, Cl, or I), alkali salt of C₁₂-C₁₈ fatty acid.

In order to achieve the technical object, oleyl amine, octyl amine, hexadecyl amine, and octadecylamine are one of the C₆-C₂₀ alkyl amine and may be included as the C₆-C₂₀ alkyl amine other than amines listed above.

In still yet another preferred embodiment, cetyl trimethyl ammonium bromide, octyl trimethyl ammonium bromide, and dodecyltrimethylammonium bromide are one of alkyl trimethyl ammonium salts and may be included as the alkyl trimethyl ammonium salt other than the above bromides.

In a further preferred embodiment, oleic acid, linoleic acid, lauric acid, myristic acid, palmitic acid, and stearic acid are one of alkali salts of C₁₂-C₁₈ fatty acids and may be included as the alkali salts of C₁₂-C₁₈ fatty acids other than the acids.

In another aspect, the present invention may provide a ceria-zirconia nano complex further comprising: a phospholipid-polyethylene glycol (PEG) layer absorbing the surface of the surfactant layer of the ceria-zirconia nanoparticles including the surfactant layer.

In a preferred embodiment, the ceria-zirconia nano complex may have a diameter of 5 to 30 nm.

In yet another aspect, the present invention provides an antioxidant including the ceria-zirconia nano complex.

In another preferred embodiment, the ceria-zirconia nano complex may have a diameter of 5 to 30 nm.

In a preferred embodiment, reactive oxygen species removed by the antioxidant may be any one of hydrogen peroxide (H₂O₂), superoxide anion (O₂—), and hydroxyl radical (OH.).

In still another aspect, the present invention provides a sepsis treating agent including the ceria-zirconia nano complex.

In a preferred embodiment, the ceria-zirconia nano complex may have a diameter of 1 to 5 nm.

In still yet aspect, the present invention provides a preparing method of ceria-zirconia nanoparticles comprising: (a) preparing a mixed solution by mixing a cerium precursor, a zirconium precursor, and a surfactant; (b) dispersing the mixed solution; (c) generating a colloid solution by heating the dispersed solution; and (d) precipitating only the ceria-zirconia nanoparticles by centrifuging the colloid solution.

In a preferred embodiment, the cerium precursor in step (a) may be selected from a group consisting of cerium (III) acetylacetonate hydrate, cerium (III) acetate hydrate, cerium (III) carbonate hydrate, cerium (IV) hydroxide, cerium (III) fluoride, cerium (III) chloride, cerium (III) chloride heptahydrate, cerium (III) bromide, cerium (III) iodide, cerium (III) nitrate hexahydrate, cerium (III) sulfate, cerium (III) sulfate hydrate, and cerium (IV) sulphate.

In another preferred embodiment, the zirconium precursor in step (a) may be selected from a group consisting of zirconium (IV) acetylacetonate hydrate, zirconium (IV) acetate hydrate, zirconium (IV) carbonate hydrate, zirconium (IV) hydroxide, zirconium (IV) fluoride, zirconium (IV) chloride, zirconium (IV) chloride octahydrate, zirconium (IV) bromide, zirconium (IV) iodide, zirconium (IV) oxynitrate hydrate, zirconium (IV) sulfate hydrate, and zirconium (IV) sulfate.

In still another preferred embodiment, the surfactant in step (a) may be at least one selected from a group consisting of C₆-C₂₀ alkyl amine, alkyl trimethyl ammonium salt ((CH₃)₃RNX, wherein R is C₈-C₂₅, X is Br, Cl, or I), alkali salt of C₁₂-C₁₈ fatty acid.

In yet another preferred embodiment, C₆-C₂₀ alkyl amine in step (a) may be oleyl amine, octyl amine, hexadecyl amine, and octadecylamine and further, include other amines other than the amines.

In still yet another preferred embodiment, the alkyltrimethyl ammonium salt in step (a) may be cetyl trimethyl ammonium bromide, octyl trimethyl ammonium bromide and dodecyl trimethyl ammonium bromide, and further include other bromides.

In yet another preferred embodiment, the alkali salt of C₁₂-C₁₈ fatty acids in step (a) may be oleic acid, linoleic acid, lauric acid, myristic acid, palmitic acid and stearic acid, and further include other acids.

The preparing method may further include cooling and then washing the colloid solution between steps (c) and (d).

In a preferred embodiment, in the dispersing in step (b), the mixed solution may be dispersed by using a sonicator at room temperature for 10 to 20 minutes.

In another preferred embodiment, the heating condition of step (c) may be constituted by a first step of heating up to 70 to 90° C. at a heating speed of 1 to 5° C./min in the heating condition of step (c); and a second step of maintaining the temperature of 70 to 90° C.

In yet another preferred embodiment, the second step may be maintained for 3 to 30 hours.

In still another preferred embodiment, in the cooling condition, the cooling may be performed up to 10 to 30° C.

In still yet another aspect, the present invention provides a preparing method of a ceria-zirconia nano complex comprising: (a) preparing a first mixed solution by mixing a first dispersion including ceria-zirconia nanoparticles and a second dispersion including phospholipids-PEG; (b) obtaining powder including the ceria-zirconia nanoparticles by evaporating the first mixed solution to remove a solvent; (c) preparing a second mixed solution by adding the powder to water; and (d) separating the second mixed solution with a filtrate by passing through a filter media having a plurality of filtering holes.

In a preferred embodiment, in the step (a), the concentration of the first dispersion including the ceria-zirconia nanoparticles may be 10 mg/ml.

In another preferred embodiment, in the step (a), the concentration of the second dispersion including the phospholipids-PEG may be 10 mg/ml.

In still another preferred embodiment, each solvent of the first dispersion and the second dispersion may be any one of chloroform (CHCl3), dichloromethane, pentane, hexane, heptane, cyclohexane, ethyl acetate, tetrahydrofuran, diethyl ether, and trichloroethylene.

In yet another preferred embodiment, in the step (d), the size of the plurality of filtering holes of the filter media may be 0.1 to 1.0 μm.

In still yet another preferred embodiment, the preparing method may further comprise: (e) washing the remaining phospholipids-PEG among the particles filtered on the surface of the filter media, after step (d).

### BRIEF DESCRIPTION OF THE DRAWINGS

FIG. 1 illustrates a characteristic of a ceria-zirconia nano complex synthesized in Example 3 of the present invention.

FIG. 2 illustrates a process of synthesizing ceria-zirconia nanoparticles and the ceria-zirconia nano complex which are synthesized in Examples 2 and 3 of the present invention.

FIG. 3 illustrates an image for the surface of the ceria-zirconia nano complex, as TEM and STEM images of the ceria-zirconia nano complex.

FIG. 4 illustrates an assay result using an X-ray diffractometry (XRD), a photoelectron spectroscopy (XPS), and a particle size analyzer (DLS) of the ceria-zirconia nano complex.

FIG. 5 illustrates a result according to an assay of an energy dispersive spectroscopy (EDS) of the ceria-zirconia nano complex.

FIG. 6 illustrates in vitro data of cell mortality of the ceria-zirconia nano complex.

FIG. 7 illustrates in vivo data of mortality by comparing an experimental group and a control group of ceria-zirconia with respect to mortality in an acute sepsis disease model experiment.

FIG. 8 is a structural diagram illustrating a preparing method of the ceria-zirconia nanoparticles.

FIG. 9 is a structural diagram illustrating a preparing method of the ceria-zirconia nano complex.

### DETAILED DESCRIPTION OF THE PREFERRED EMBODIMENTS

Hereinafter, the present invention will be described with reference to the accompanying drawings. As those skilled in the art would realize, the described embodiments may be modified in various different ways, all without departing from the spirit or scope of the present invention. Parts which are not related with the description are omitted in order to clearly describe the present invention in the drawings and like reference numerals designate like elements throughout the specification.

Throughout this specification and the claims that follow, when it is described that an element is “coupled” to another element, the element may be “directly coupled” to the other element or “electrically coupled” to the other element through a third element. In addition, unless explicitly described to the contrary, the word “comprise” and variations such as “comprises” or “comprising”, will be understood to imply the inclusion of stated elements but not the exclusion of any other elements.

Terms used in the present application are used only to describe specific exemplary embodiments, and are not intended to limit the present invention. Singular expressions used herein include plurals expressions unless they have definitely opposite meanings. In the present application, it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations, in advance.

Hereinafter, Examples of the present invention will be described in detail with reference to the accompanying drawings.

### Example 1. Preparing Method of Ceria Nanoparticles

1 mmol (0.4 g) of a cerium (III) acetate hydrate (Sigma-Aldrich) and 12 mmol (3.2 g) oleyl amine (approximately 80 to 90% of C₁₈ content, Acros Organics) were added to 15 ml of xylene (98.5%, Sigma-Aldrich). The prepared solution was dispersed for 15 minutes at room temperature by using a sonicator and then heated up to 90° C. at a velocity of 2° C./min. While the solution was vigorously stirred at 90° C., 1 ml of deionized water was injected to the solution, and the solution was changed from off-white to cloudy yellow, and this means that reaction was initialized. The obtained mixture was aged for 3 hours at 90° C. to obtain a transparent yellow colloid solution and the obtained colloid solution was cooled at room temperature. Thereafter, the precipitate was washed well with 100 ml of acetone by using a centrifugation method, and the washed ceria nanoparticles was stored in chloroform at a concentration of 10 mg/ml so as to be dispersed well.

### Example 2. Preparing Method of Ceria-Zirconia Nanoparticles

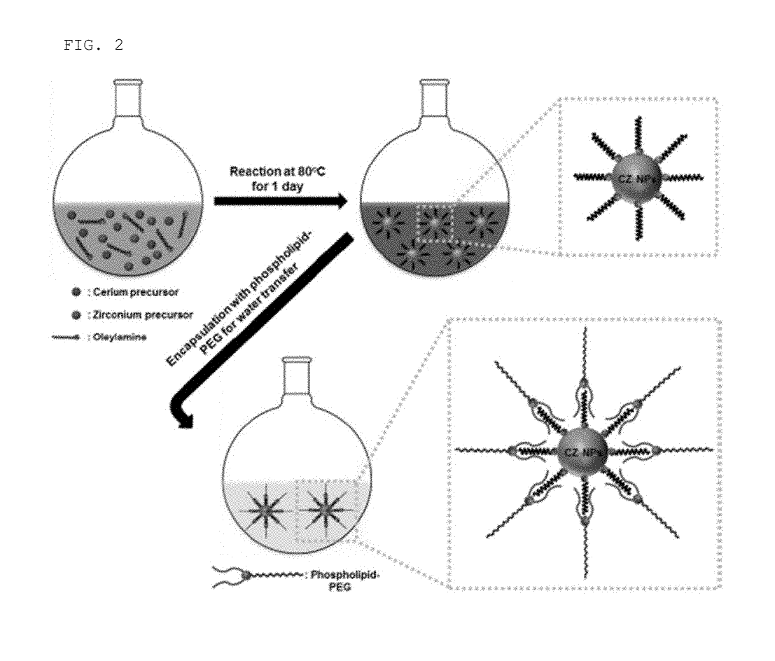

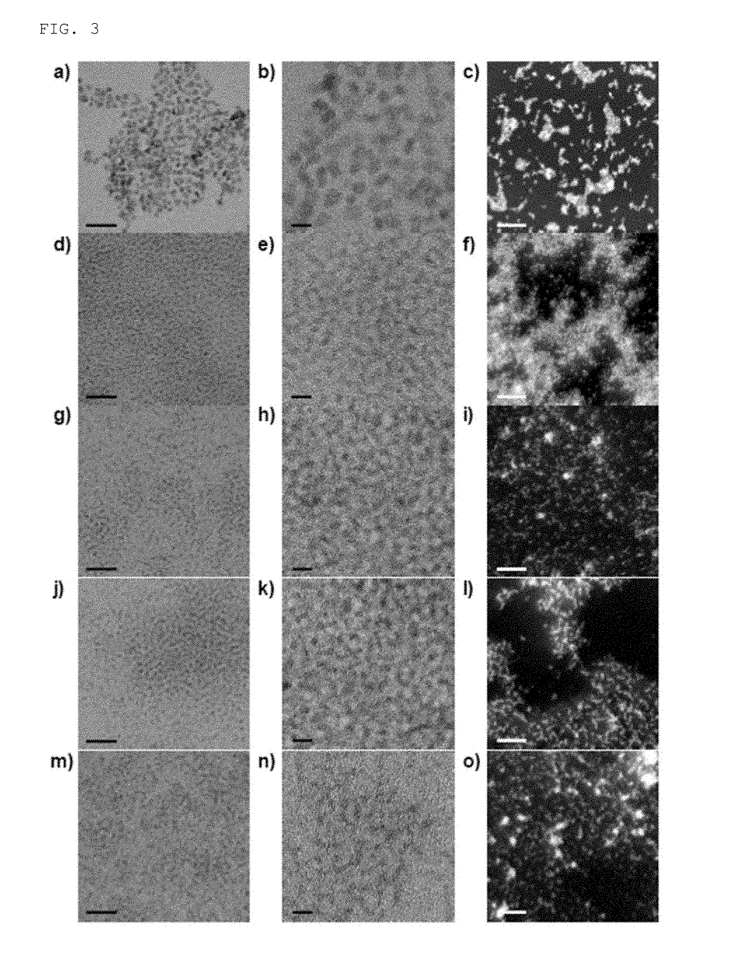

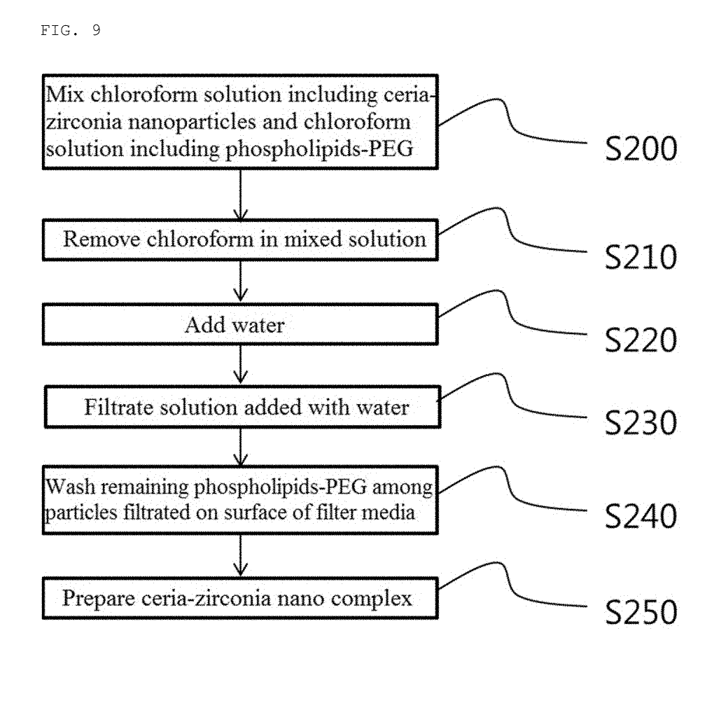

Total 0.5 g of a mixture of a cerium (III) acetylacetonate hydrate (Sigma-Aldrich) and a zirconium (IV) acetylacetonate hydrate (Sigma-Aldrich) was added to 15 ml of oleyl amine (approximately 80 to 90% of C18 content, Acros Organics) with a molar ratio of cerium (III):zirconium (IV)=100:0 to 20:80 (see FIGS. 2 and 8). The prepared solution was dispersed for 15 minutes at room temperature by using a sonicator and then heated up to 80° C. at a velocity of 2° C./min. Thereafter, the solution was maintained at 80° C. and aged for one day to obtain a dark brown colloid solution, and the solution was cooled at room temperature. Thereafter, the precipitate was washed well with 100 ml of acetone by using a centrifugation method, and the washed ceria-zirconia nanoparticles was stored in chloroform at a concentration of 10 mg/ml so as to be dispersed well.

In the present invention, the ceria-zirconia (CeₓZr₁-xO₂) nanoparticles or the ceria-zirconia nano complex, a sample including the nanoparticles or the nano complex, and the like, when x was 1, it was defined as 10CZ, when x was 0.7, it was defined as 7CZ, when x was 0.4, it was defined as 4CZ, and when x was 0.2, it was defined as 2CZ. Further, 2CZ, 4CZ, and 7CZ were commonly defined as CZ NPs. Simple ceria nanoparticles were defined as Ce NPs.

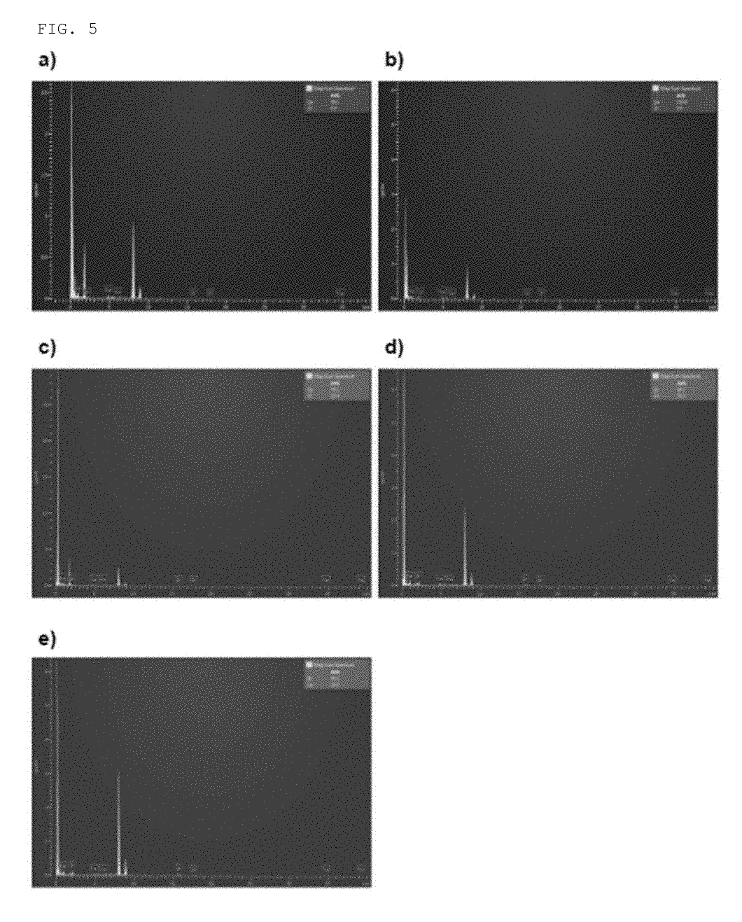

The energy dispersive spectrometry (EDS) is one of an optional function attached to a scanning electron microscope (SEM), and when an X-ray detector detects an X-ray signal to convert the X-ray signal into an electronic signal, the X-ray detector measures the converted electronic signal by using a pulse processor and then determines the detected X-ray energy. Whether the synthesis of the ceria-zirconia nanoparticles according to Example 2 is correctly performed can be verified by analyzing the determined X-ray data (see FIG. 5). Referring to an EDS graph, (a) is Ce NPs, (b) is 10CZ, (c) is 7CZ, (d) is 4CZ, and (e) is 2CZ, and whether the synthesis is correctly performed can be verified based on a composition table.

### Example 3. Preparing Method of Ceria-Zirconia Nano Complex

In order to improve biocompatibility of the ceria-zirconia nanoparticles, phospholipids PEGylation was performed (see FIGS. 2 and 9). The PEGylation is a technique of absorbing a safe biocompatible polymer as polyethylene glycol (PEG) on an interface of medicines or other targets. 10 mg/ml of chloroform added with 5 ml of ceria-zirconia nanoparticles was mixed with 10 mg/ml of chloroform including 10 ml of 1,2-distearoyl-sn-glycero-3-phosphoethanol amine-N-[methoxy(polyethylene glycol)-2000] (mPEG-2000, Avanti Polar Lipids Inc) with a ratio of 1:2. Chloroform as a solvent was evaporated by using a rotary evaporator and incubated at 70° C. in a vacuum drier to remove all of the remaining chloroform. Thereafter, 5 ml of water was added to the generated powder to prepare a transparent colloid suspension. The suspension was filtered by using a filter with a size of 0.4 m to remove a large amount of mPEG-2000 through ultra-centrifugation. The ceria-zirconia nano complex encapsulated with the refined phospholipids-PEG was dispersed in distilled water.

### Example 4. Superoxide Dismutase (SOD) Mimetic Activity Assay

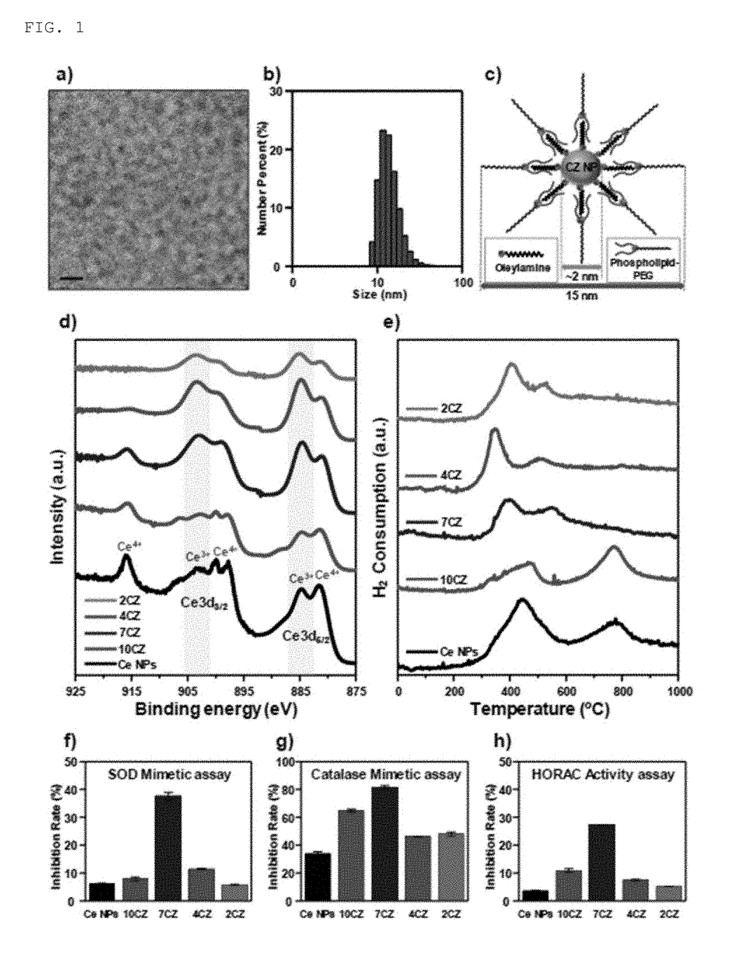

The SOD is an enzyme that catalyzes disproportionation reaction that converts superoxide ions into oxygen and hydrogen peroxide. It is known that an antioxidant defense mechanism is performed in almost all cells that are exposed in oxygen. A removal of the superoxide ions was performed in an SOD assay kit (Sigma-Aldrich), 20 μl of each sample was mixed with 160 μl of a WST-1 standard solution (0.125 mM), and then 20 μl of a xanthine oxidase solution was added to microplate wells, respectively, and the reaction occurred. When the microplate wells were incubated for 20 minutes at 37° C., absorbance of 450 nm was observed through a multiple plate reader (Victor X4, Perkin-Elmer) (FIG. 1F). Since an inhibition rate of the superoxide ions may be calculated as the deployment of the color is reduced, the absorbance of 450 nm becomes standards of quantification.

### Example 5. Catalase Mimetic Activity Assay

Hydrogen peroxide quenching is performed by using an Amplex® red hydrogen peroxide/peroxide enzyme assay kit (molecular probes, Inc). When a horseradish peroxide (HRP) enzyme and a Amplex®red reactant react with hydrogen peroxide, a red fluorescent material of resorufin is generated (see FIG. 1G).

In this case, red fluorescein of resorufin reflects the degree of peroxide in a solution (excitation and emission are performed at maximum 571 nm and 585 nm). The hydrogen peroxide and the sample are mixed, and until the final concentrations are 10 mM and 0.25 mM, respectively, 50 μL of a hydrogen peroxide solution is prepared. When 50 μL of Amplex® red/HRP standard solution is added to the microplate wells, respectively, the reaction starts. The sample needs to be incubated under a condition of 25° C. in a non-light state, and thereafter, a fluorescent material is measured by using a multiple plate reader (Victor X4, Perkin-Elmer).

### Example 6. Hydroxyl Radical Oxidation-Preventing Capacity (HORAC) Assay

A hydroxyl radical collection activity may be analyzed by using a HORAC assay kit (Cell Biolaps, Inc. USA) (see FIG. 1H). When a hydroxyl radical introducing agent and a fenton reagent are mixed, the hydroxyl radical is easily generated, the fluorescent material may be continuously verified by using the generated hydroxyl radical. The inhibition rate of the hydroxyl radical of the solution may be calculated by measuring the intensity (excitation is 480 nm and emission is 530 nm) of fluorescent light. 20 μL of each sample (the final concentration of 0.125 mM) was mixed with 140 μL of a fluorescent material 1X. When the mixture was incubated at 25° C. for 30 minutes, 20 μL of a hydroxyl introducing agent 1X and 20 μL of a fenton reagent were continuously injected to microplate wells, respectively. When the plate was shaken for 15 seconds and then incubated at 25° C. for 30 minutes, the fluorescent material may be observed by using a multiple plate reader Victor X4.

### Example 7: Cell Viability Experiment Using Tert-Butyl Hydroperoxide (t-BHP) (In Vitro)

20,000 RAW 264.7 cells were first implanted in a plate with 96 wells and then incubated at 37° C. for 24 hours. Thereafter, the plate was washed with a phosphate buffer saline (PBS) solution (a solution used as a suspension of an organism, a tissue, or an organ in which the life cannot be maintained long only in a saline), and 10 mL of Ce NPs and 7CZ (the final concentrations were 0, 0.01, and 0.02 mM) were treated in the plate coated with RAW 264.7 cells and incubated at 37° C. for 24 hours. The incubating cells were washed with the PBS solution and finally treated with 10 mL of tBHP (the final concentration was 0.4 mM) and further incubated for 2 hours. After incubating, 10 mL of a 3, (4,5-dimethylthiazol-2-yl)-2.5-diphenyl tetrazolium bromide (MTT) solution (the concentration was 5 mg/ml) was treated in the wells, respectively, and then incubated at the same temperature for 2 hours. Thereafter, the washed cells were treated with 200 mL of a DMSO solution, and the viability of the cells was observed by using a multiple plate reader Victor X4.

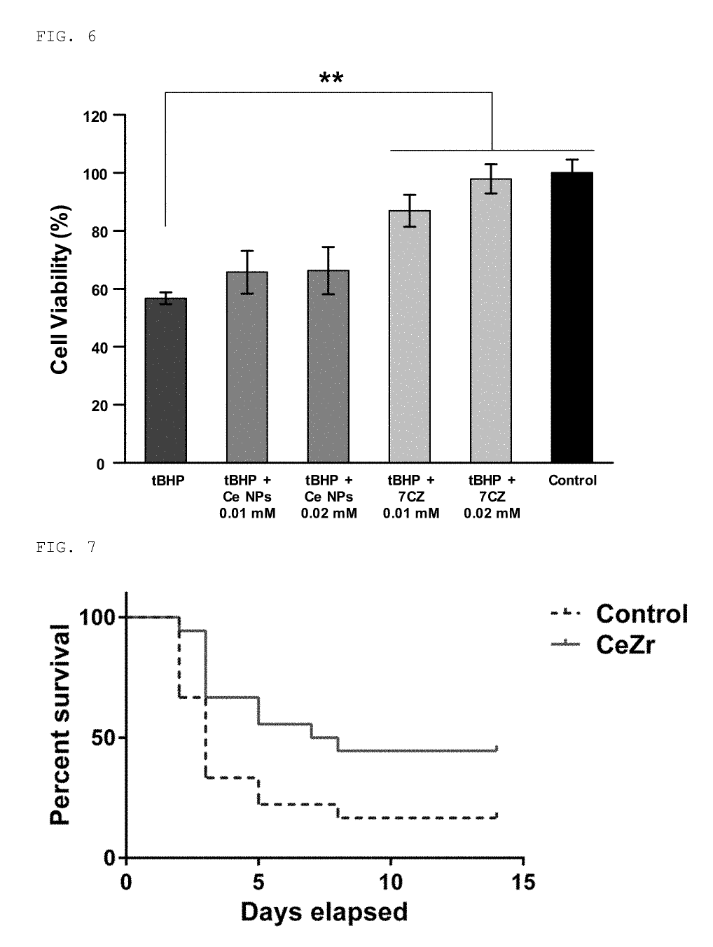

It can be seen that the survival capacity of a 7CZ treating group at a concentration of 0.02 mM is most excellent on the basis of the assay (see FIG. 6).

### Example 8. Cecal Ligation and Puncture (CLP) Assay (In Vivo)

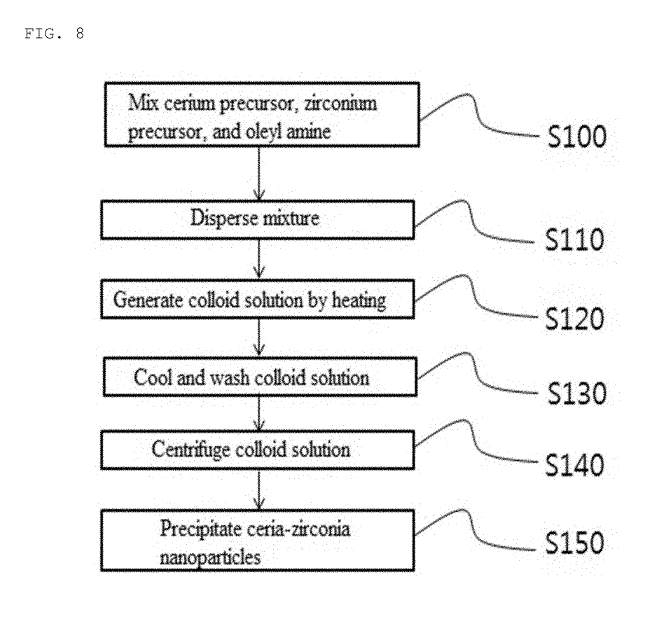

In an In vivo experiment, a cecal ligation and puncture (CLP) model (acute sepsis model) that may actually similarly imitate the sepsis was prepared (see FIG. 7). Thereafter, 18 mice were injected with the PBS solution (a control group) and 18 mice were injected with CZ NPs (an experimental group) and then, viability for 14 days was observed.

TABLE 1

| | | | | Reduction | |
|---|---|---|---|---|---|
| | Core size | HD diameter | | Temperature (° C.) | |
| sample | (nm) | (nm) | Ce³⁺ (%) | LT Peak | HT Peak |
| Ce NPs | 3.55 ± 0.37 | 15.06 ± 4.18 | 30.08 | 446.3 | 784.8 |
| 10CZ | 2.1 ± 0.21 | 13.38 ± 4.05 | 30.42 | 483.3 | 779.1 |
| 7CZ | 2.08 ± 0.22 | 14.21 ± 4.27 | 52.61 | 397.9 | 549.7 |
| 4CZ | 2.19 ± 0.17 | 16 ± 4.54 | 59.81 | 347.8 | 513.2 |
| 2CZ | 2.21 ± 0.18 | 11.01 ± 3.3 | 63.31 | 403.9 | 531.3 |

On the basis of the XPS assay, when binding energy was 884.5 and 903 eV, Ce³⁺ peaks were observed (see FIG. 1D and Table 1). The XPS assay means that photoelectrons were emitted from a sample when X rays having constant energy was irradiated to the sample, and binding energy of the photoelectrons was determined by measuring kinetic energy of the photoelectrons. The binding energy was a unique property of atoms emitting the photoelectrons and thus, the atoms may be analyzed by the binding energy.

On the basis of Ce³⁺ peaks illustrated in the XPS assay, as more Zr⁴⁺ ions were bound to the ceria-zirconia nano complex, it can be seen that more Ce³⁺ of the ceria-zirconia nano complex is present, Further, generally, as the size of the particle is deceased, more Ce³⁺ is present in the nanoparticles. As Ce³⁺ is increased, it can be seen that a reduction effect of superoxide anion (O₂—) and hydroxyl radical (OH.) is more increased.

Further, in order to compare an effect according to the size of the particle, Ce NPs and 10CZ which are samples without including Zr⁴⁺ are analyzed.

According to an existing research result, when the synthesis process for generating particles is performed in an aqueous state, it was known that the particles included more Ce³⁺ For this reason, 10CZ has a smaller size, but 10CZ was synthesized in an organic solution state, and since the synthesis of Ce NPs was performed in the aqueous state, relatively, a ratio of 10CZ and Ce³⁺/Ce⁴⁺ is almost similar.

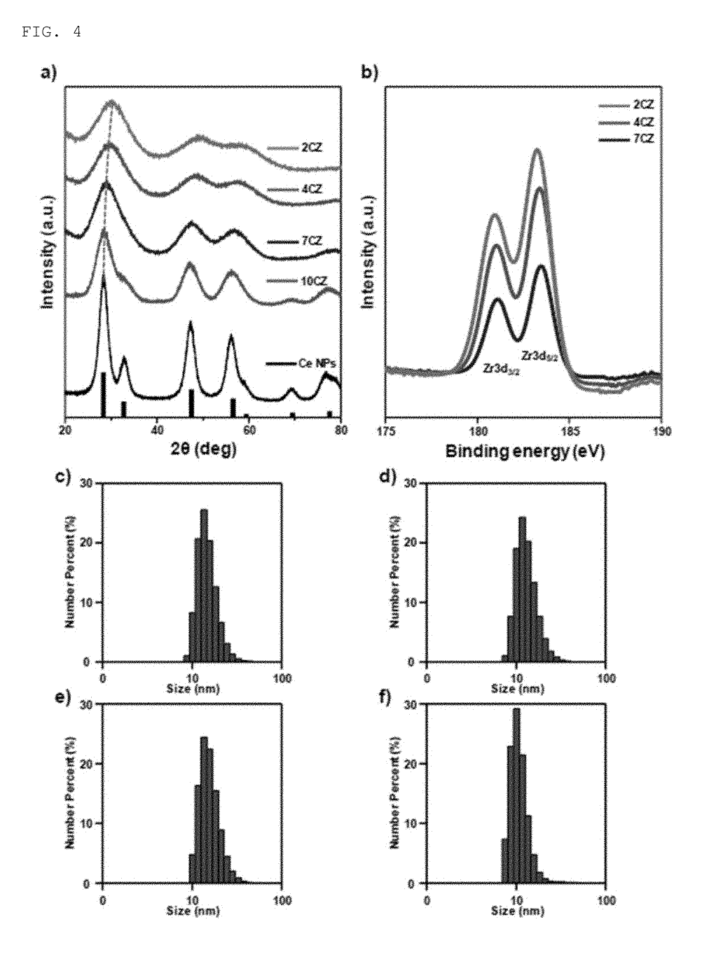

A difference according to the reaction state condition can be seen according to the XRD assay result (see FIG. 4A).

The XRD assay is an X-ray diffractometry, and when X rays collide with the crystal, some of the crystal are diffracted and the diffraction angle and strength are unique on the material structure, and information related with a kind and an amount of a crystalline material included in the sample can be seen by using the diffracted X rays. As such, the assay method for obtaining the information on the structure of the crystalline material is the X-ray diffractometry (XRD).

According to the XRD assay, 2CZ, 4CZ, and 7CZ have the same crystalline structure as Ce NPs and 10CZ. Further, as the content of Zr⁴⁺ ions is increased, a diameter of the Zr⁴⁺ ions is small and tetragonality is further increased. Thus, it is determined that whether Ce³⁺ is included according to a reaction condition state as the existing research result is valid.

The effect on Ce⁴⁺ reduction due to Zr⁴⁺ may be more accurately determined by analyzing a H₂-TPR graph (see FIG. 1E) During reduction reaction of Zr⁴⁺, it can be seen that H₂ consumption peaks LT peak and HT peak was enhanced at a low temperature and a high temperature (see Table 1). When the samples Ce NPs and 10CZ without including Zr⁴⁺ are compared with the samples 2CZ, 4CZ, and 7CZ including Zr⁴⁺ and two peaks of the samples including Zr⁴⁺ are compared with those of the Ce NPs sample, it may be observed that the samples are rapidly changed. It can be seen that Zr⁴⁺ helps in entirely inducing a reduction reaction of Ce⁴⁺→Ce³⁺.

As a result, under the condition that the size of the particle is very small, the ratio of Ce³⁺/Ce⁴⁺ is high, and rapid Ce³⁺ reproduction for removing reactive oxygen species is mild, the CZ NPs may be easily prepared.

In order to compare reactive oxygen species removal capability of CZ NPs and Ce NPs, superoxide anion (O₂—), hydrogen peroxide (H₂O₂), and hydroxyl radical (OH) are selected from the reactive oxygen species and inspected at room temperature.

In the reactive oxygen species removal capability according to the size of the particle, when comparing Ce NPs and 10CZ, it is determined that 10CZ with a smaller size has more excellent reactive oxygen species removal capability by all of the preformed assay results.

When comparing 7CZ and 10CZ, it can be seen that catalase mimetic activity capacity of 10CZ having the high content of Ce⁴⁺ is enhanced. The reason is that when hydrogen peroxide is removed, the catalase mimetic activity capacity is largely influenced by Ce^{4+.} Even though there is a limit of a material having oxidation-reduction reaction with respect to the superoxide anion and the hydroxyl radical, 4CZ and 2CZ including less Ce ions had a similar inhibition rate as compared with the assay of Ce NPs and 10CZ. This is a result supporting that the ratio of Ce³⁺/Ce⁴⁺ increasing according to the Zr⁴⁺ content and the rapid reproduction speed to Ce³⁺ play the most important role in the reduction reaction of the superoxide anion and the hydroxyl radical.

According to all the assay result, it can be seen that 7CZ has the most excellent reactive oxygen species removal capability. Furthermore, physiologically, it can be seen that as Zr is appropriately added to the ceria nanoparticles, Zr⁴⁺ has an excellent effect on removal of the reactive oxygen species.

As a result of analyzing a survival curve result using a CLP model (see Example 8 and FIG. 7), when the accumulated viability is verified, in a control group of injecting the PBS solution for 14 days, only three mice were survived, and in an experimental group of injecting 7CZ, eight mice were survived. It can be seen that the death rate of the control group is 83.3% and the death rate of the experimental group is 55.6%. Further, in the CLP model, it can be seen that CZNPs are dispersed around cerium where sepsis is caused and act to a local portion. As a result, it can be seen that the ceria zirconia nanoparticles accurately have an effect as a sepsis treating agent in vivo.

In this experiment, CZ NPs was administrated only once to reduce the death rate to 27.7%. Even though the CZ NPs do not solve pathogens as a cause of infection, it can be seen that enhancing the viability plays a very important role to suppress the excessive bio-error reaction by the reactive oxygen species removal capacity of CZ NPs. Further, since the CZ NPs have the reproduction effect, the CZ NPs have the reactive oxygen species (ROS) suppressing effect even only one administration.

Further, in the survival graph of the CLP model, in the control group, the death rate is most high for initial 2 to 3 days and thereafter, it may be observed that objects are slowly died. There is no large difference from the 7CZ experimental group after 3 days. It is suggested that it is important that the CZ NPs are rapidly treated in an initial infection, and it is more effective that the CZ NPs rapidly react as compared with the Ce NPs. Like the in vitro experiment (see FIG. 6) above, in the initial infection reaction, it can be seen that a sepsis treating effect of the CZ NPs is more excellent.

According to the exemplary embodiments of the present invention, the ceria-zirconia nanoparticles and the ceria-zirconia nano complex can efficiently remove reactive oxygen species. Therefore, the ceria-zirconia nano complex of the present invention may have an effective treating effect as an antioxidant and a sepsis treating agent.

The effects of the present invention are not limited to the above effects and it should be understood that the effects include all effects inferable from the configuration of the invention described in the detailed description or claims of the present invention.

While the present invention has been described with respect to the specific embodiments, it will be apparent to those skilled in the art that various changes and modifications may be made without departing from the spirit and scope of the invention as defined in the following claims.

1. A ceria-zirconia nanoparticles comprising:
a core layer consisting of particles made of ceria-zirconia; and
a surfactant layer formed by binding a surfactant on the surface of the core layer.

2. The ceria-zirconia nanoparticles of claim 1, wherein the core layer has a diameter of 1 to 5 nm.

3. The ceria-zirconia nanoparticles of claim 1, wherein the ceria-zirconia is expressed as CeₓZr₁₋ₓO₂ and x is 0.1 to 1.

4. The ceria-zirconia nanoparticles of claim 1, wherein the surfactant is at least one selected from a group consisting of C₆-C₂₀ alkyl amine, alkyl trimethyl ammonium salt ((CH₃)₃RNX, wherein R is C₈-C₂₅, X is Br, Cl, or I), alkali salt of C₁₂-C₁₈ fatty acid.

5. A ceria-zirconia nano complex comprising:
a surfactant layer included in the ceria-zirconia nanoparticles of claim 1; and
phospholipid-polyethylene glycol (PEG) layer absorbing an interface of the surfactant layer.

6. The ceria-zirconia nano complex of claim 5, wherein the ceria-zirconia nano complex has a diameter of 5 to 30 nm.

7. An antioxidant including the ceria-zirconia nano complex of claim 5.

8. The antioxidant of claim 7, wherein reactive oxygen species removed by the antioxidant is any one of hydrogen peroxide (H₂O₂), superoxide anion (O₂—), and hydroxyl radical (OH.).

9. A sepsis treating agent including the ceria-zirconia nano complex of claim 5.

10. A preparing method of ceria-zirconia nanoparticles comprising:
(a) preparing a mixed solution by mixing a cerium precursor, a zirconium precursor, and a surfactant;
(b) dispersing the mixed solution;
(c) generating a colloid solution by heating the dispersed solution; and
(d) precipitating only the ceria-zirconia nanoparticles by centrifuging the colloid solution.

11. The preparing method of claim 10, wherein the cerium precursor in step (a) is selected from a group consisting of cerium (III) acetylacetonate hydrate, cerium (III) acetate hydrate, cerium (III) carbonate hydrate, cerium (IV) hydroxide, cerium (III) fluoride, cerium (III) chloride, cerium (III) chloride heptahydrate, cerium (III) bromide, cerium (III) iodide, cerium (III) nitrate hexahydrate, cerium (III) sulfate, cerium (III) sulfate hydrate, and cerium (IV) sulphate.

12. The preparing method of claim 10, wherein the zirconium precursor in step (a) is selected from a group consisting of zirconium (IV) acetylacetonate hydrate, zirconium (IV) acetate hydrate, zirconium (IV) carbonate hydrate, zirconium (IV) hydroxide, zirconium (IV) fluoride, zirconium (IV) chloride, zirconium (IV) chloride octahydrate, zirconium (IV) bromide, zirconium (IV) iodide, zirconium (IV) oxynitrate hydrate, zirconium (IV) sulfate hydrate, and zirconium (IV) sulfate.

13. The preparing method of claim 10, wherein the surfactant in step (a) is at least one selected from a group consisting of C₆-C₂₀ alkyl amine, alkyl trimethyl ammonium salt ((CH₃)₃RNX, wherein R is C₈-C₂₅, X is Br, Cl, or I), alkali salt of C₁₂-C₁₈ fatty acid.

14. The preparing method of claim 10, further comprising:
cooling and then washing the colloid solution between steps (c) and (d).

15. The preparing method of claim 10, wherein in the dispersing in step (b), the mixed solution is dispersed by using a sonicator at room temperature for 10 to 20 minutes.

16. The preparing method of claim 10, wherein the heating condition of step (c) includes a first step of heating up to 70 to 90° C. at a heating speed of 1 to 5° C./min in the heating condition of step (c); and a second step of maintaining the temperature of 70 to 90° C.

17. The preparing method of claim 16, wherein the second step is maintained for 3 to 30 hours.

18. The preparing method of claim 14, wherein in the cooling condition,
the cooling is performed up to 10 to 30° C.

19. A preparing method of a ceria-zirconia nano complex comprising:
(a) preparing a first mixed solution by mixing a first dispersion including ceria-zirconia nanoparticles and a second dispersion including phospholipids-PEG;
(b) obtaining powder including the ceria-zirconia nanoparticles by evaporating the first mixed solution to remove a solvent;
(c) preparing a second mixed solution by adding the powder to water; and
(d) separating the second mixed solution with a filtrate by passing through a filter media having a plurality of filtering holes.

20. The preparing method of claim 19, wherein in the step (a), the concentration of the first dispersion including the ceria-zirconia nanoparticles and the concentration of the second dispersion including the phospholipids-PEG are 10 mg/ml.

21. The preparing method of claim 19, wherein in the step (a), each solvent of the first dispersion and the second dispersion is any one of chloroform (CHCl₃), dichloromethane, pentane, hexane, heptane, cyclohexane, ethyl acetate, tetrahydrofuran, diethyl ether, and trichloroethylene.

22. The preparing method of claim 19, wherein in the step (d), the size of the plurality of filtering holes of the filter media is 0.1 to 1.0 μm.
